(19) United States
(12) Patent Application Publication
(10) Pub. No.: US 2013/0103092 A1
(43) Pub. Date: Apr. 25, 2013

(54) PRE-ASSEMBLED SPINAL CONSTRUCT AND METHOD OF USE

(71) Applicant: Rodney R. Ballard, Lakeland, TN (US)

(72) Inventor: Rodney R. Ballard, Lakeland, TN (US)

(73) Assignee: WARSAW ORTHOPEDIC, INC., Warsaw, IN (US)

(21) Appl. No.: 13/281,173

(22) Filed: Oct. 25, 2011

(51) Int. Cl. A61B 17/70; A61B 17/88

(52) U.S. Cl. 606/265; 606/279

(57) ABSTRACT

A pre-assembled spinal construct is provided which includes an elongated member, a plurality of heads spaced apart at select positions around the elongated member, each of the plurality of heads defining an enclosure configured to receive a first securing member for securing the heads around the elongated member and each head aligned for coupling with a second securing member. A method for treating a spine utilizing the pre-assembled spinal construct of the present application is also provided, the method including the steps of providing a pre-assembled spinal construct, implanting the pre-assembled spinal construct into the spine of a patient in need thereof and coupling the pre-assembled spinal construct to at least two bone fasteners.

### TECHNICAL FIELD

The present disclosure generally relates to medical devices for the treatment of spinal disorders, and more particularly to a spinal construct, that can be pre-assembled and snapped onto pre-existing bone screws.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatments of these spinal disorders include discectomy, laminectomy, fusion and implantable prosthetics. As part of these surgical treatments, connecting elements such as spinal rods are often used to provide stability to a treated region. During surgical treatment, one or more rods may be attached to the exterior of two or more vertebral members by using pedicle screws. The pedicle screws are generally placed two per vertebra, one at each pedicle on either side of the spinous process, and serve as anchor points for the spine rods. Clamping elements adapted for receiving a spinal rod are then used to join the spinal rods to the screws. The clamping elements are commonly assembled to the head of the pedicle screws.

One type of clamping element mounted to a pedicle screw has a housing resembling a saddle connected to the screw. The housing includes a U-shaped channel for receiving a spinal rod therein. After the pedicle screw has been inserted into bone and the spinal rod is positioned in the U-shaped channel, a set screw is threaded into internal threads of the housing for securing the spinal rod in the U-shaped channel.

Surgeons have frequently encountered considerable difficulty when attempting to insert spinal fixation devices. For example, during the surgical procedure, surgeons are frequently unable to efficiently and adequately place the spinal rod into the U-shaped heads of the pedicle bone screws because the U-shaped heads of the screws are often not aligned with one another due to a curvature in the spine and the different orientations of the pedicle screws. The spinal rods are often bent in multiple planes to couple the pedicle screws to the rod, which may lead to weaker connections with the rod. These problems also result in significantly longer surgical procedure time, thereby increasing the likelihood of complications associated with surgery.

It would therefore be desirable to address the shortcomings of the prior art by providing a (pre-assembled spinal construct which addresses the difficulty in properly positioning spinal rods and rod-capturing assemblies. These pre-assembled spinal constructs minimize manipulation of the many parts associated with these complex devices, and decrease surgical steps, reduce inventory parts. These spinal constructs can, in some embodiments, also be attached to already existing pedicle screws from previous surgery and at the same time, they can reduce anesthesia time and patient bleeding.

### SUMMARY OF THE INVENTION

Accordingly, in some embodiments, the present application provides a pre-assembled spinal construct which includes an elongated member, a plurality of heads spaced apart at select positions around the elongated member, each of the plurality of heads defining an enclosure configured to receive a first securing member for securing each head onto the elongated member and wherein each head is aligned for coupling with a second securing member.

in one embodiment, the enclosure of each head includes a top surface defining a first opening configured to receive the first securing member, a bottom surface defining a second opening configured to receive the second securing member, two lateral surfaces opposite each other and connecting the top and the bottom surfaces, each lateral surface defining a third opening and fourth opening, these openings configured to slidably receive the elongated member, and anterior and posterior surfaces connecting the top and the bottom surfaces.

in another embodiment the elongated member of the pre-assembled spinal construct is solid or hollow, uniform in size and shape and has a cross-section selected from spherical, elliptical or hexagonal configurations.

In yet another embodiment, the elongated member of the pre-assembled spinal construct varies in size and shape along its length, and has an angled or curved orientation.

In another aspect, the first securing member is selected from a set screw, a pin, or a rod, and the second securing member is a bone fastener selected from a pedicle screw, or a compression screw, a pin, or a shaft that secures to bone. It is envisioned that the first securing member may include any securing member having a means that provides friction and/or clamping sufficient to secure each head onto the elongated member, such as, for example, a ¼″ type fastener.

In an alternate embodiment the enclosure defined by the head contains biological material including osteogenic growth factors.

In some embodiments the present application also provides a method for treating a spine, the method including the steps of providing an elongated member, at least two heads configured to be slidably received over the elongated member and at least two bone fasteners, securing the at least two heads at selected positions around the elongated member, each of the at least two heads configured for alignment with the at least two bone fasteners to form a pre-assembled spinal construct, implanting the pre-assembled spinal construct into the spine of a patient and coupling the pre-assembled spinal construct to the at least two bone fasteners.

### BRIEF DESCRIPTION OF THE DRAWINGS

The present disclosure will become more readily apparent from the specific description accompanied by the following drawings, in which:

FIG. 1 is a schematic of one particular embodiment of the spinal construct in accordance with the principles of the present disclosure;

FIG. 2 is a perspective view of the head of the spinal construct shown in FIG. 1;

FIG. 3 is a sectional view of the spinal construct shown in FIG. 1;

FIG. 4 is a side view of a set screw of the spinal construct shown in FIG. 1; and

FIG. 5 is a side elevation view of the shall and head of a pedicle screw.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure. Like reference numerals indicate similar parts throughout the figures.

### DETAILED DESCRIPTION OF THE INVENTION

The exemplary embodiments of the spinal construct and methods of use disclosed are discussed in terms of medical devices for the treatment of musculoskeletal spinal disorders and more particularly, in terms of a pre-assembled spinal construct for delivery to a surgical site and a method for treating a spine. It is envisioned that the pre-assembled spinal construct and methods of use disclosed provide stability and maintain structural integrity while reducing the number of steps in a surgical procedure thereby increasing the efficiency and accuracy of the surgeon with many beneficial results to the patient.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures, of such disorders, such as, for example, discectomy, laminectomy, fusion, bone graft and implantable prosthetics. It is contemplated that the present disclosure may be employed with other osteal and bone related applications, including those associated with diagnostics and therapeutics. It is further contemplated that the disclosed pre-assembled spinal construct may be employed in a surgical treatment with a patient in a prone or supine position, employing a posterior, lateral or anterior approach. The present disclosure may be employed with procedures for treating the lumbar, cervical, thoracic and pelvic regions of a spinal column.

In some embodiments the present application may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

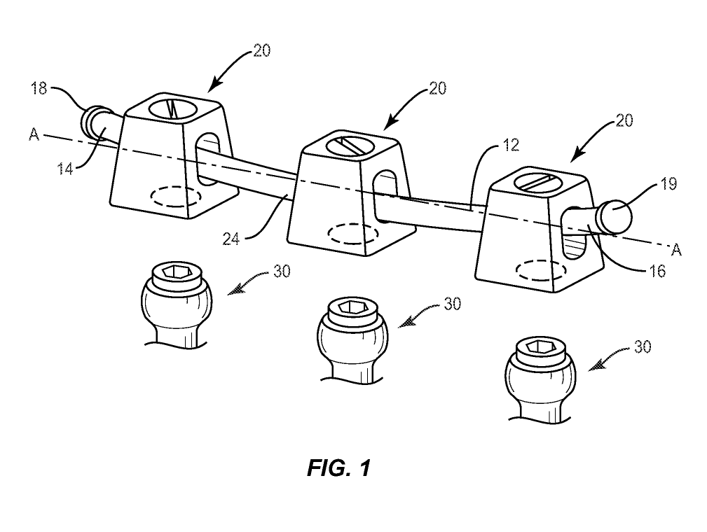

The following discussion includes a description of a pre-assembled spinal construct, related components and exemplary methods of employing the spinal construct in accordance with the principles of the present disclosure. Alternate embodiments are also disclosed. Reference now be made in detail to the exemplary embodiments of the present disclosure, which are illustrated in the accompanying figures. Turning now to FIGS. 1-5, there are illustrated components of a pre-assembled spinal construct in accordance with the principles of the present disclosure.

The components of the pre-assembled spinal construct are fabricated from materials suitable for medical applications, including metals, polymers, ceramics, biocompatible materials and/or their composites, depending on the particular application and/or preference of a medical practitioner. For example, the elongated member, discussed below, of the spinal construct can be fabricated from materials such as titanium, thermoplastics such as polyaryletherketone (PAEK) including PEEK, PEKK and PEK, carbon-PEEK. composites, PEEK-BaSO₄ polymeric rubbers, biocompatible materials such as polymers including plastics, metals, ceramics and composites thereof rigid polymers including polyphenylene, polyamide, polyimide, polyetherimide, polyethylene, epoxy; and different sections of an elongated member, as discussed below, may have alternative material composites to achieve various desired characteristics such as strength, rigidity, elasticity, compliance biomechanical performance, durability and radiolucency or imaging preference.

With reference to FIG. 1 in an embodiment of the present application, there is provided a pre-assembled spinal construct 10, including an elongated member 12 a plurality of heads 20 spaced apart at discrete positions around the elongated member, and a plurality of bone fasteners 30, each of the plurality of heads configured for alignment and coupling with the plurality of bone fasteners 30. These can be snap-fittings, friction fittings, threading or the like for engagement with heads 20.

The elongated member 12 has two opposite ends 14 and 16, each end having an end stop element 18 and 19 attached thereto. End stop elements 18 and 19 are configured to prevent disengagement of the heads from the elongated member. In an embodiment each end stop element has a diameter which is larger than that of elongated member 12.

In one embodiment, the elongated member is a spherical rod, uniform in size and shape having a smooth surface over which the heads can be slidably received. In some embodiments, the circumference of the rod is in a range from about 3.0 mm to about 7.0 mm.

Other shapes such as elliptical, or shapes having planar sidewalls extending along the length of the elongated member are also contemplated. The elongated member may have different cross-sectional shapes than those shown and described. For example, the cross-section of an elongated member having planar sidewalls can be square, rectangular, hexagonal, or a D-shaped. The elongated member can be solid or hollow for its entire length or only portions thereof.

It is also contemplated that in some embodiments, the elongated member may be configured so that orientation in one direction provides one set of stabilizing properties to the vertebrae, white orienting the elongated member in the other direction would provide a second set of stabilizing properties.

It should be noted that the elongated member can be made of elastic or semi-elastic materials in parts or in its entirety. Exemplary elastic materials include polyurethane, silicone, silicone-polyurethane, polyolefin rubbers, hydrogels, and the like. The elastic materials can be resorbable, serni-resorbable, or non-resorbable. Exemplary inelastic materials include polymers, such as polyetheretherketone (PEEK), polyetherketoneketone (EKK), and polylactic acid materials (PLA and PLDLA), metals, such as titanium, NITINOL, and stainless steel, and/or ceramics, such as calcium phosphate and alumina. Further, the elongated member can be solid, hollow, semi-hollow, braided, woven, mesh, porous, or combinations thereof. The elongated member can also be reinforced or semi-reinforced and have a variable rigidity. As a result, the elongated member can be curved or include a curved portion 24 defined between ends 14 and 16. For example, depending on the shape of the elongated member, the curved portion can be disposed at an angle relative to a longitudinal axis A-A from about 0 degrees to about 60 degrees.

Also, different curved portions can be located between the at least two or more heads. Increasingly, the elongated member can be manufactured to perform differently at discrete positions along its length based on the material and the orientation of the elongated member. In a pre-assembled spinal construct, a precise orientation of the different sections of the elongated member can be engineered before a surgical procedure begins, thereby saving not only procedure steps and surgery time but also achieving more precise performance characteristics.

The spinal construct (e.g., elongated member) can be fabricated using a number of fabrication techniques, such as injection molding, machining and milling from a solid stock material. For example, the spinal construct (e.g., elongated member) can be manufactured from, for example, machining and milling, extrusion and die cutting, injection molding, transfer molding and/or cast molding. One skilled in the art, however, will realize that such materials and fabrication methods suitable for assembly and manufacture, in accordance with the present disclosure, would be appropriate.

In FIG. 1, the geometry of the elongated member is shown as uniform in size and shape. However, it is contemplated that the elongated member can vary in size and geometry along its length. For example, the elongate member could be tapered from one end 14 to the other end 16 or have multiple geometries along its length.

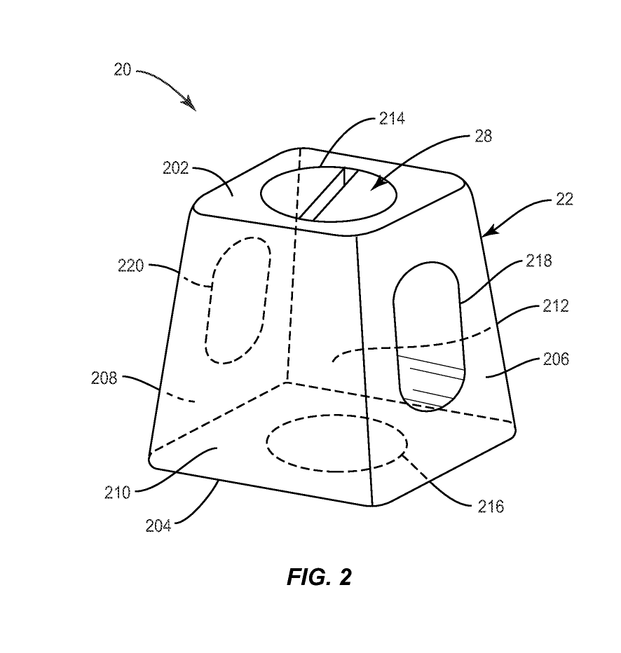

With reference to FIG. 2, each head 20 is configured to slide over the elongated member in order to be securely positioned thereon at discrete locations selected for alignment and coupling to pre-placed bone screws and to accommodate other shapes present in a patient's spine, To that end, each head 20 defines an enclosure 22 which is generally comprises atop surface 202, a bottom surface 204, lateral surfaces 206 and 208, connecting to the top and bottom surfaces, an anterior surface 210 and a posterior surface 212 (shown in phantom), also connecting the top and bottom surfaces, 202 and 204. The top surface 202 defines an opening 214, which opening is configured to receive the first securing member 28. The bottom surface 204 defines an opening 216 (shown in phantom in FIG. 2) configured to receive a second securing member 30. Lateral surfaces 206 and 208 define openings 218 and 220 (shown in phantom in FIG. 2), respectively, configured to receive slidably elongated member 12. Openings 218 and 220 can have the same or different shapes varying with the geometry of the elongated member. In one embodiment, openings 218 and 220 can have a spherical or elliptical circumference. Anterior and posterior surfaces 210 and 212 generally do not contain any openings. However, in one embodiment in which the head contains biological material, each or both the anterior and posterior surfaces 210 and 212 define one or more small openings (not shown in FIG. 2) useful for the release of the biological material.

The spinal construct may be used with growth factors including, but not limited to, members of the fibroblast growth factor family, including acidic and basic fibroblast growth factor (FGF-1 and FGF-2) and FGF-4, members of the platelet-derived growth factor (PDGF) family, including PDGF-AB, MGR-BB and PDGF-AA; EGFs; the TGF-β superfamily, including TGF-β1, 2 or 3; osteoid-inducing factor (OIF); angiogenin(s); endothelins; hepatocyte growth factor or keratinocyte growth factor; members of the bone morphogenetic proteins (BMP's) BMP-1, BMP-3, BMP-2; OP-1, BMP-2A, BMP-2B, or BMP-7; HBGF-1 or HBGF-2; growth differentiation factors (GDF's); members of the hedgehog family of proteins, including indian, sonic and desert hedgehog; ADMP-1; other members of the interleukin (IL) family; or members of the colony-stimulating factor (CSF) family, including CSF-1, G-CSF, and GM-CSF, or isoforms thereof; or VEGF, NELL-1 (neural epidermal growth factor-like 1), CD-RAP (cartilage-derived retinoic acid-sensitive protein) or combinations thereof.

In some embodiments, the spinal construct may be used with osteogenic proteins. Exemplary osteogenic proteins include, but are not limited to OP-1, OP-2, OP-3, BMP-2, BMP-3, BMP-3b, BMP-4, BMP-5, BMP-6, BMP-9, .BMP-10, BMP-11, BMP-12, BMP-13, BMP-14, BMP-I5, GDF-1, GDF-2, GDF-3, GDF-5, GDF-6, GDF-7, GDF-8, GDF-9, GDF-10, GDF-11, GDR-12, CDMP-1, CDMP-2, CDMP-3, DPP, Vg-1, Vgr-1, 60A protein, NODAL, UNWIN, SCREW, ADMP, NEURAL, and TGF-beta. As used herein, the terms “morphogen, ” “bone morphogen, ” “BMP, ” “osteogenic protein” and “osteogenic factor” embrace the class of proteins typified by human osteogenic protein 1 (hOP-1).

Exemplary growth factors include, but are not limited to, members of the transforming growth factor beta family, including bone morphogenetic protein 2 (BMP-2); bone morphogenetic protein 4 (BMP-4); and transforming growth factors beta-1, beta-2, and beta-3 (potent keratinocyte growth factors). Other useful members of the transforming growth factor beta family include BMP-3, BMP-5, BMP-6, BMP-9, DPP, Vg1, Vgr, 60A protein, GDF-1, GDF-3, GDF-5, GDF-6, GDF-7, CDMP-1, CDMP-2, CDMP-3, BMP-10, BMP-11, BMP-13, BMP-15, Univin, Nodal, Screw, ADMP, Neural, and amino acid sequence variants thereof Other growth factors include epidermal growth factor (EGF), which induces proliferation of both mesodermal and ectodermal cells, particularly keratinocytes and fibroblasts; platelet-derived growth factor (PDGF), which exerts proliferative effects on mesenchymal cells; fibroblast growth factor (FGF), both acidic and basic; and insulin-like growth factor 1 (IGF-1) or 2 (IGF-2), which mediate the response to growth hormone, particularly in bone growth. Further growth factors include osteogenic proteins. A particularly preferred osteogenic protein is OP-1, also known as bone morphogenetic protein 7 (BMP-7). OP-1 is a member of the transforming growth factor beta gene superfamily.

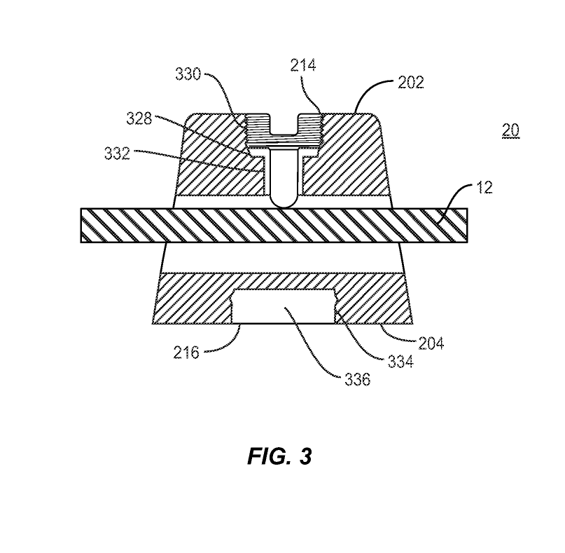

With reference to FIG. 3, at opening 214 of top surface 202, head 20 has a first recess 328 configured to receive a first securing member 28 (FIG. 1), such as for example a set screw. Recess 328 has a threaded portion 330 and a tapered portion 332, both configured to receive the first securing member 28 as further described below. At opening 216 of bottom surface 204, head 20 has a second inner recess 334 that is adapted to receive a second securing member 30 (FIG. 1). The second inner recess 334 has generally a threaded portion 336, which can mate to the second securing member 30 (FIG. 1) as further described below. Elongated member 12 can be received in the head.

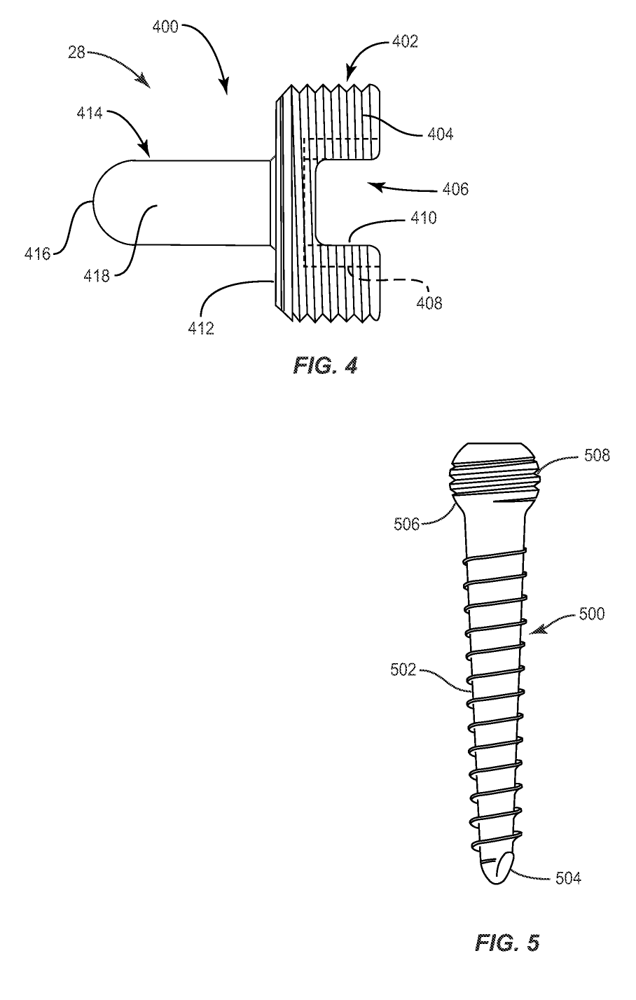

With reference to FIG. 4, securing member 28 is preferably a set screw 400 generally comprised of a head portion 402 and an alignment member 414 integral therewith. Head portion 402 includes a threaded outer surface 404, a recess 406 and a front face 412. Threaded outer surface 404 has threads that mate with the threads of threaded section 330 of head 20. In a preferred embodiment of the present application, head portion 402 has an outer diameter of approximately 5.0 mm or greater. Recess 406 can have internal threads 408. Moreover, notch 410 is formed at the upper end of head portion 402. Recess 406, threads 408 and notch 410 are dimensioned to interface with a conventional instrument (e.g., a screw driver with a draw rod) for rotating set screw 400 into head 20 in order to secure the head onto elongated member 12. It should be understood that head portion 402 may take other forms suitable for interfacing with other types of instruments for rotating set screw 400.

Alignment member 414 is integrally attached to head portion 402 at front face 412. According to an embodiment, alignment member 414 includes a generally cylindrical elongated portion 418 and a full spherical radius tip 416 at the distal end thereof. It will be appreciated elongated portion 418 may have other suitable geometries (e.g., hex, square, etc.). Moreover, tip 416 may be flat or have other suitable geometries.

It will be appreciated that set screw 400 may have alternative configurations. In this regard, the alignment member 414 could have a non-circular shape or a sharp tip. In another embodiment alignment member 414 may be omitted from set screw 400, thus providing a set screw with a generally planar front face. Moreover, the alignment member 414 could be omitted and replaced with a hole extending through the entire set screw. In this case, the alignment member could be located on the driving instrument.

in a preferred embodiment of the present application, set screw 400 has a diameter of approximately 6.0 mm or greater. It will be appreciated that in an alternative embodiment of the present application, set screw 400 could be replaced by other suitable locking mechanisms. As illustrated in FIG. 1 and FIG. 3, the first securing member 28 operates to secure head 20 onto the elongated member 12 at selected positions around the elongated member.

As shown in FIG. 5, the second securing member 30 can be a bone screw such as a pedicle screw 500 having a threaded shaft 502 and a tip 504 for insertion into the spine. A crown 506 of the pedicle screw 500 is located opposite from the tip 504 and includes a generally spherical or convex profile with a helical thread 508 extending circumferentially around the screw crown 506. The pedicle screw 500 includes a hexagonal or other shaped socket (not shown) by which an appropriate tool may be utilized by a surgeon to screw the pedicle screw 500 into the patient's spine or other bone structure. The pedicle screw 500 is preferably polyaxial and is designed to threadably engage with head 20 at recess 334 in which the elongated member is secured by set screw 28 present at recess 328. As described above, head 20 is configured to couple with pedicle screw 500 to allow for a variety of different configurations for the elongated member 12. In some embodiments, the crown 506 of the bone screw does not need to have threading.

Another advantage of the spinal construct of the present application is that as a result of pre-assembling, head 20 can be designed to couple with any bone screw already present in the pedicle from a previous surgery wherein the head might have had a conventional U-shaped channel in which the spinal rod was secured by a set screw. Head 20 is also removable. The ability to remove specific components such as the head from a conventional already installed pedicle screw and attach to it the (pre-assembled spinal construct according to the present application offers significant benefits to both surgeon and patient, The threaded engagement between the head 20 of the present application and pedicle screw 500 allows for head 20 to be removed, replaced, adjusted and/or reattached to the screw as needed. As such, the pedicle screw insertion is not restricted by the geometry or mechanics of the spinal construct.

Both set screw 400 and pedicie screw 500 are preferably made of implant grade titanium alloy (Ti-6Al-4V (ELI) per ASTM F-136), or other biocompatible material, such as stainless steel, carbon fiber reinforced polyaryletherketone composites and the like.

It is also contemplated that the pre-assembled spinal construct of the present application can be used with pedicle screws coated with an osteoconductive material such as hydroxyapatite and/or osteoinductive agent such as a bone morphogenic protein for enhanced bone fixation. The elongated member 12 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of elongated member 12.

In use, to treat the affected section of the spine, a medical practitioner obtains access to a surgical site including vertebrae in any appropriate manner, such as through incision and retraction of tissues. It is envisioned that the pre-assembled spinal construct can be used in any existing surgical method or technique including open surgery, mini-open surgery, minimally invasive surgery and percutaneous surgical implantation, whereby the vertebrae are accessed through a micro-incision, or sleeve that provides a protected passageway to the area. Once access to the surgical site is obtained, the pre-assembled spinal construct is snapped onto in-place fasteners such as bone screws preferably pedicle screws. Utilizing a pre-assembled spinal construct saves many surgical steps that would have been required had the spinal construct been assembled in situ element by element. Moreover, based on patient information available prior to surgery, the spinal construct of the present application can be engineered with great precision ordinarily not available during the surgical procedure.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A pre-assembled spinal construct comprising an elongated member, a plurality of heads spaced apart at selected positions around the elongated member, each of the plurality of heads defining an enclosure configured to receive a first securing member for securing each head around the elongated member, each head aligned for coupling with a second securing member.

2. A spinal construct of claim 1, wherein the enclosure of each head comprises; a top surface defining a first opening configured to receive the first securing member; a bottom surface defining a second opening configured to receive the second securing member; two lateral surfaces opposite each other and connecting the top surface and the bottom surface, each lateral surface defining a third opening and fourth opening each configured to slidably receive the elongated member; and an anterior surface and a posterior surface connecting the top surface and the bottom surface.

3. A spinal construct of claim 1, wherein the elongated member is uniform in size and. shape and has a cross-section in a spherical, elliptical or hexagonal configuration.

4. A spinal construct of claim 1, wherein the elongated member varies in size and shape along its length or comprises an angled orientation or is curved.

5. A spinal construct of claim 1, wherein the elongated member has opposite ends, each end containing a stop end.

6. A spinal construct of claim 1, wherein the first securing member is selected from a set screw or a pin.

7. A spinal construct of claim 1, wherein the second securing member is a bone fastener selected from a pedicle screw, or a compression screw or a shaft

8. A spinal construct of claim 1, wherein each head has a different shape.

9. A spinal construct of claim 1 wherein the enclosure is defined by the head and further comprises biological material, the anterior and posterior surfaces further comprise apertures for release of a biological material,

10. A spinal construct of claim 9, wherein the biological material comprises a growth factor.

11. A spinal construct of claim 10, wherein the growth factor comprises bone morphogenic protein.

12. A method for treating a spine, the method comprising:
providing an elongated member, at least two heads configured to be slidably received over the elongated member and at least two bone fasteners;
securing the at least two heads at selected positions around the elongated member, each of the at least two heads configured for alignment with the at least two bone fasteners to form a pre-assembled spinal construct;
implanting the pre-assembled spinal construct into the spine of a patient; and
coupling the pre-assembled. spinal construct to the at least two bone fasteners.

13. A method of claim 12, wherein the elongated member is uniform or varies in size and shape.

14. A method of claim 12, wherein each of at least two heads further comprises a top surface defining an opening for securing a rod, wherein the securing member comprises a set screw or pin.

15. A method of claim 12, wherein the bone fastener comprises a pedicle screw.

16. A method of claim 12, wherein the pre-assembled spinal construct contains biological material comprising a growth factor.

17. A method of claim 16, wherein the growth factor comprises bone morphogenic protein.
